(19) United States
(12) Patent Application Publication
(10) Pub. No.: US 2024/0148525 A1
(43) Pub. Date: May 9, 2024

(54) ORTHOPAEDIC SOCKET AND METHOD FOR ADJUSTMENT THEREOF

(71) Applicant: OTTOBOCK SE & CO. KGAA, Duderstadt (DE)

(72) Inventors: Mario KOPPE, Duderstadt (DE); Stefan REINELT, Duderstadt (DE); Lars Benjamin FINKE, Duderstadt (DE); Leonard VIER, Duderstadt (DE); Johannes SIEGEL, Duderstadt (DE)

(21) Appl. No.: 18/549,715

(22) PCT Filed: Mar. 29, 2022
(86) PCT No.: PCT/EP2022/058254

§ 371 Date: Sep. 8, 2023

(30) Foreign Application Priority Data
Mar. 29, 2021 (DE) 10 2021 107 816.4

(51) Int. Cl. A61F 2/80; A61F 2/70

(52) U.S. Cl. CPC A61F 2/80; A61F 2/70; A61F 2002/503

(57) ABSTRACT

The invention relates to an orthopaedic socket for bearing on and for receiving a limb stump or a limb, having at least one socket wall (2), a proximal access opening (3), a distal end (4) and at least one fastening device (5) which is arranged for fastening at least one distal orthopaedic component (6) to the socket (1), wherein at least one stiffening element (40) is arranged or formed on or in the socket wall (2), said stiffening element being assigned an adjustment device (30) and by means of which a stiffness of the socket wall (2) is reversibly variable at least in certain regions.

The invention relates to an orthopedic socket, in particular a prosthesis socket, for bearing on and for receiving a limb stump or a limb, with at least one socket wall, a proximal access opening, a distal end, and at least one fastening device which is arranged to fasten at least one distal orthopedic component to the socket. The invention also relates to a method for adjusting such an orthopedic socket.

Orthopedic sockets, in particular prosthesis sockets, are used to receive limb stumps or limbs and can have a large number of configurations. In one configuration of a socket, an impression is taken of the limb or the limb stump on which the socket is to be fitted. If necessary with addition of material, this impression is used to model the socket, for example from a thermoplastic or a fiber composite material. The socket wall is generally a closed wall and dimensionally stable and has a proximal access opening. Devices for fastening further components, for example joints or the like, are arranged, in particular incorporated, at the distal end or distal end region.

Alternatively, the sockets can be produced in an additive manufacturing process.

In order to produce a better fit and an adaptation to different contours or volumes, prosthesis sockets are produced with an open cross section or from several components that are displaceable relative to one another. The components are positioned relative to one another and braced relative to one another and fixed in the respective position via motors or via tension devices. By releasing the tension means or a closure, the socket volume serving to accommodate the limb or the limb stump can be increased. This takes place, for example, when putting on and taking off the socket or in order to relieve the limb or the limb stump, for example when sitting.

The sockets, in particular prosthesis sockets, hold the further orthopedic components securely on the limb or the limb stump and also ensure that the further orthopedic components are correctly aligned with the limb or the limb stump. For this purpose, it is necessary for the orthosis socket or prosthesis socket to have sufficient stiffness and stability to ensure reliable guiding of the orthopedic component fastened to it.

Fixed, rigid abutment surfaces, such as the ramus contact, are a necessity for suitable positioning of the socket on the limb or limb stump. However, such rigid abutment surfaces are disadvantageous in terms of comfort.

The object of the present invention is to make available an orthopedic socket and a method for adjusting an orthopedic socket, with which an increase in wearing comfort is achieved while at the same time maintaining precise guidance of orthopedic components.

According to the invention, this object is achieved by an orthopedic socket with the features of the main claim and by a method with the features of the additional independent claim. Advantageous embodiments and developments of the invention are disclosed in the dependent claims, the description and the figures.

In the orthopedic socket for bearing on and for receiving a limb stump or a limb, having at least one socket wall, a proximal access opening, a distal end, and at least one fastening device which is arranged for fastening at least one distal orthopedic component to the socket, provision is made that at least one stiffening element is arranged or formed on or in the socket wall, which stiffening element is assigned an adjustment device and by means of which a stiffness of the socket wall is reversibly variable at least in certain regions. With such a socket, it is possible that certain surfaces or regions of the socket wall have the necessary stiffness or rigidity only when this is necessary. In particular, in different situations of use, the stiffness can be adjusted in certain regions in order to ensure either precise guidance and stable support of the limb or the limb stump or to provide sufficient flexibility so that the socket is comfortable to wear.

The stiffening element works in combination with the socket wall and serves to change the stiffness in the corresponding regions. The stiffening element is in particular an additional component on a socket, which component is fastened, arranged, formed or integrated thereon.

In one embodiment of the socket, the stiffening element is mounted displaceably in or on the socket wall, such that it is able to be positioned in the immediate vicinity of the regions whose stiffness is to be changed. A bearing on the socket wall, in particular on the outside of the socket wall, facilitates accessibility, for example in order to carry out repairs or to make adjustments. An arrangement in the socket wall uses the socket wall as such for protection and to form a housing, so that the stiffening element is protected from external influences. The bearing or arrangement within the socket wall enables a compact configuration and in particular a smooth-surfaced contour of the outer socket wall.

In one variant, the stiffening element is assigned a motor drive, e.g. via an adjustment element which is arranged between the drive or the adjustment device and the stiffening element. By way of the drive or the adjustment device, it is possible to twist, move, bend or otherwise displace or manipulate the adjustment element and/or the stiffening element in order to achieve the desired change in stiffness in the respective region of the socket wall. A linear actuator is also regarded as a motor drive, likewise switchable magnetic devices that cause a displacement.

In one embodiment, the adjustment element is designed as a gear wheel, roller, crank, lever, tension means, rotary element, piston, thrust means and/or valve. Tension devices are components or parts that transmit tensile forces. The tension means can be rigid or flexible; in particular, tension means are flexible and non-elastic, in particular flexible under pressure and rigid under tension. In principle, there is also the possibility that the tension means have an elasticity, such that they can also serve as an energy accumulator and force limiter at the same time. Rotary elements that are moved around a pivot point or an axis of rotation can be designed, for example, as wheels, rollers, shafts, spindles, screws or also twistable components such as cords or chains and also rods. Pistons are moved linearly and displace a medium. In an alternative embodiment, the pistons are designed as rotary pistons and displace the respective medium not by a linear movement but by a rotational movement about an axis of rotation of the rotary piston. Thrust means are components that transmit compressive forces, for example rods, connecting rods, rigid chains, or other pressure-resistant components, optionally in combination with elastic components that enable force accumulation and force limitation. If the stiffness is adjusted by supply or discharge of fluids, then, in one embodiment, valves are designed as switching valves, adjustment valves or other adjustable flow limiters with which the flow rate can be adjusted or a flow channel can be blocked. The force accumulators are designed in particular as springs, compressible fluids, accumulators for electrical energy or accumulators for thermal energy.

In one embodiment, the adjustment device is designed as an electromagnet, pump, electric motor, heater, cooling element, piezoelectric element, electroactive polymer, energy accumulator, manually operable device or voltage source. The adjustment device makes it possible to adjust or change the corresponding stiffening element or, if necessary, the adjustment element, in order thereby to adjust and change the stiffness in the region of the socket wall. Either a stiffening element or adjustment element can be displaced via the electromagnet, or a magnetic field can be generated in order to change the viscosity of a magnetorheological liquid. A fluid or another pumpable medium can be pumped into or out of a volume via a pump, in order to change the stiffness of the volume and thus of the region of the prosthesis socket in which the volume is located. For example, if liquid is pumped into a volume, the increased internal pressure usually increases the stiffness in the region in which the volume is located. Stiffness can also be increased if the volume is filled with structural elements and a vacuum is applied. Due to the negative pressure, the side walls are pressed against the structural elements and increase the stiffness of the socket in this region. Mechanical components can be adjusted via an electric motor, for example elements with different area moments of inertia can be twisted or aligned in a different way in order to enable adaptation of the stiffness. A motor can also drive pumps, displace or wind up force transmission devices and adjust valves. By means of the heater, for example a resistance heater, the viscosity and the stiffness can be changed by heating specific regions. Conversely, the strength can be increased by cooling certain regions.

In one embodiment, at least one stiffening element, which is arranged in or on the socket wall, is assigned to the adjustment element and/or the adjustment device. The stiffening element is displaced, activated, changed in its nature, brought into a different orientation, placed in a different relationship to other elements, in order thereby to cause a change of stiffness in the respective region in which or on which the stiffening element is arranged. The stiffening element can be designed, for example, as a fluid volume, shape memory element, corrugated body, accordion element, toothed body, spring, foam body, phase change material, foldable element, slide, insert, damper or electro-adhesion element.

In one variant, the adjustment device has an actuator which can be activated and deactivated via a control device assigned to it, the control device being coupled to at least one sensor and activating or deactivating the actuator on the basis of sensor data. Instead of or in addition to a sensor, the control device can also be coupled to a switch or a switching device, via which it is possible to activate the actuator manually. The switching device can be designed, for example, in the form of a remote control or a mobile phone or a mobile data processing device that can be coupled to the control device. The coupling can take place via a cable connection or wirelessly, for example via a radio interface. For this purpose, corresponding interfaces are assigned to or arranged on the mobile data processing device and the control device. If the adjustment device is controlled automatically on the basis of sensor data, the sensor data are evaluated in the control device or alternatively in an external evaluation unit. The control device has the necessary components such as communication interface, memory, processors, energy accumulators and, optionally, signal amplifiers and integrated circuits. The control device can be coupled to one or more sensors, the sensor or sensors being selected from a group of sensors that includes a temperature sensor, an acceleration sensor, an inertial measurement unit (IMU), a spatial position sensor, an inclination sensor, a force sensor, an optical sensor, a collector electrode, a pressure sensor, a strain gauge and a resistance sensor. The optical sensor is used, for example, to record an existing oxygen saturation level or to measure the blood sugar level, and myoelectric signals are recorded via the collector electrodes. The resistance sensor measures, for example, the moisture on the inside of the socket wall and can use this to draw conclusions regarding the stress or perspiration of the user of the socket.

In one embodiment, a receptacle for the stiffening element, possibly an adjustment element and/or the adjustment device, is arranged in the socket wall or on the socket wall, so that the adjustment element, the adjustment device or the stiffening element can be subsequently and/or exchangeably fastened to or in the socket wall.

In one embodiment, the socket wall surrounds the limb or the limb stump over the entire circumference thereof and thus has a closed cross section. The embodiment with a circumferentially closed cross section increases the overall structural strength of the socket, and the comfort requirements can be adapted accordingly via the regions with the adjustable stiffness.

In one embodiment, the stiffening element and the adjustment device or the adjustment element, the adjustment device and the stiffening element are combined as a stiffening module. This stiffening module can be exchangeably arranged on or in the socket wall in order to be able to exchange existing components, retrofit them or adapt them to the respective user. The stiffening module can be industrially prefabricated and tested in advance with regard to the safety regulations to be observed, watertightness and other requirements. The stiffening module can be fixed in a reversibly detachable manner on the socket wall, for example by form-fit or force-fit engagement.

With the socket, it is possible to integrate industrially prefabricated, modular components into individual prosthesis sockets or orthosis sockets, with the stiffness of these components being able to be reversibly adjusted, as a result of which the stiffness of the socket or of the socket wall can be adapted at particular points or in particular regions, which can be individually selected. For orthoses, the term socket is also used for rails, shells, corsets and/or a pelvic cage. By virtue of the possibility of being able to reversibly change the stiffness of individual contact surfaces in the socket, the overall characteristics of the socket can be adapted according to the situation. With such a socket, it is no longer necessary to reach a compromise, between comfort and function, that is suitable for all situations of use and that therefore usually does not afford optimal functionality in any situation.

In one embodiment, sectors with different radial elasticity are formed in a proximal edge region of the socket wall. The socket, in particular in an embodiment as a prosthesis socket, surrounds the stump or the limb substantially about the entire circumference. Seen about the circumference, a socket can be divided into different sectors that take on different tasks and are exposed to different loads in different activities or conditions. In the case of a thigh socket, this is a lateral sector, for example, which is particularly important for lateral guidance and stability when loads are applied in the direction of the center of the body. The anterior sector serves to provide support in the direction or loading direction of the front region, while the posterior sector provides support in the opposite, rear region. A medial sector takes up loads that are intended to move the socket away from the center of the body. In the case of a thigh socket, there is also the so-called ramus contact in the region of the ischium. In other socket types, in particular only the first four sectors mentioned are present; in principle, more or fewer sectors can also be arranged or formed on the socket. On account of the different loads, types of loads, load directions, load sizes and also load frequencies, it is advantageous if the sectors are designed with different elasticity in the radial direction, so that it is possible to make available socket wall portions of different stiffness in the proximal edge region, which forms an upper termination of the prosthesis socket and can extend approximately over the upper half or the upper third of the overall length of the socket. By way of the adjustment device and optionally an adjustment element, the stiffness in the individual sectors can be adjusted or changed separately and independently of one another in any desired combinations and in the necessary sizes.

The sectors can have different materials, different material thicknesses, different widths of recesses and/or different densities of recesses. If, for example, the stiffening elements are completely deactivated or set to the minimum resistance in each case, different basic stiffnesses or basic resistances to a radial displacement of the socket can be obtained in the proximal edge region on account of the basic design. The different basic stiffnesses can be provided by different materials in the different sectors. If the same materials are used, for example a plastic or a fiber-reinforced plastic, different material thicknesses can lead to different stiffnesses in the respective sectors. In addition or as an alternative to the two aforementioned measures, recesses can be provided within the sectors, which recesses influence the stiffness of the respective portion of the socket wall. One possibility for designing the recesses are slits that are introduced in the proximal-distal direction. The slits can reach as far as the upper termination of the prosthesis socket and extend in the distal direction as far as the end of the proximal edge region. The width of the recesses or slits can vary between the sectors or also within a sector. Likewise, the density of the recesses of the individual sectors can be different. The greater the number of recesses on a standardized circumferential length, the lower the stiffness, the greater the length of the recesses, the lower the stiffness, and the greater the width of the recesses, the lower the stiffness in the respective segment.

In one embodiment, the individual sectors are assigned displaceable socket wall segments as stiffening elements, which are actuated or displaced individually or also in combination with one another, in order to provide different levels of stiffness in the proximal edge region.

In one embodiment, at least one flexible region is formed in the socket wall and is coupled to a stiffening element. For example, the flexible region always allows the material within the flexible region to move radially inward in the direction of the stump or limb. When the stiffening element is relaxed or not activated, the material of the flexible region can also move in the opposite direction, so that increased play or increased mobility of the limb or of the stump can be achieved in this sector. The stiffening element is, for example, a tension device such as a belt or a cable, which is tensioned such that a radial outward displacement of the material, for example of a foam material or a textile, is not possible or is possible only to a limited extent. An absence or reduction of flexibility in a radially outward direction is advantageous under high loads, for example when performing activities quickly. Conversely, a relaxation of the tension means is advantageous during inactivity, for example, when no loads need to be taken up and when it simply has to be ensured that the socket does not detach from the limb or the stump of the user.

In the method for adjusting an orthopedic socket for bearing on and for receiving a limb stump or a limb, a socket having at least one socket wall being made available, and the socket having a proximal access opening, a distal end, and at least one fastening device which is arranged for fastening at least one distal orthopedic component to the socket, provision is made that the stiffness of the socket wall is changed at least in certain regions via at least one adjustment device and a stiffening element. The situation-dependent or freely chosen, reversible changeability of the stiffness in certain regions means that the socket as such can be adjusted in terms of its mechanical properties and that optimal functionality is provided by the situation-dependent adjustability.

In one embodiment, the stiffness is changed by a displacement of an adjustment element, an adjustment, displacement or changing of a stiffening element which is arranged on or in the socket wall, and/or by activation or deactivation of the adjustment device. The stiffening element is changed, for example, by a displacement, a compression, a change in the angle of attack of a support element, or the insertion or removal of material into and from intermediate spaces between portions that are formed by the stiffening element.

In one variant, the stiffness can be changed by twisting a spring element, wherein the spring element, depending on orientation and alignment, provides different resistances to deformation. As an alternative or in addition to twisting of a spring element, the latter can also be shifted, for example into or out of a region that is in principle intended for changing the stiffness. When a spring element is pushed into the region, the stiffness increases; when it is moved out, the stiffness of the socket wall in this region decreases. If the spring element is permanently present and is not intended to be removed from the respective region, the spring element can also be blocked, such that the resilience provided in principle by the spring element is no longer available and the socket wall is thereby stiffened in this region. The stiffening element or the adjustment element can be displaced, for example twisted, pushed in, pulled out or compressed. If a volume is present in the region of the changeable stiffness, the stiffness can be changed by filling or emptying this volume with a corresponding fluid and/or changing the viscosity of a medium that is present in the volume. Regions can be heated or cooled in order to achieve a desired change by changing the temperature-dependent bending stiffness. Stiffening elements can be brought into form-fit engagement with one another, for example by mutually corresponding toothed arrangements being brought into and out of engagement with one another.

The change in stiffness can take place on the basis of sensor data that are determined by at least one sensor and are transmitted to a control device, which in turn activates or deactivates an actuator. By way of the actuator, the necessary actions are then each initiated in order to change the stiffness, for example a heater is switched on, a cooling system is activated, a stiffening element is twisted or displaced, or another measure is taken in order to make available the desired region with the desired stiffness in each case. In particular, the stiffness is changed by twisting, shifting, tensioning, blocking and/or activating an adjustment element or the stiffening elements.

In a further development, the socket wall has sectors which are distributed about the circumference and which are changed independently of one another in terms of their stiffness. In one embodiment, the stiffness changes automatically during use of the orthopedic socket, advantageously in real time and/or as a function of the movement situation in which the user is situated, for example the gait situation if the orthopedic device is designed for the lower limbs, especially as a prosthetic leg. The data are recorded by sensors and transmitted to an evaluation and control device, and the adjustment device is activated, deactivated or modulated on the basis of these sensor data and the evaluation, in order to set the desired or required degree of stiffness. This makes it possible to set degrees of stiffness that are adapted to the respective situation, in particular distributed according to sectors, for which an adaptation to the needs or preferences of the respective user takes place in the proximal edge region. The sensor data may originate from sensors which are arranged on the socket and/or arranged on or assigned to a component fastened to the socket. If, for example, a resistance device is part of the orthopedic device on which the orthopedic socket is arranged, the sensors that are used for controlling the resistance device can also be used to detect load situations. On the basis of the respective load situations, not only can the resistance then be adjusted, the stiffness in the respective sector of the socket can also be changed.

Exemplary embodiments of the invention are explained in more detail below with reference to the accompanying figures, in which:

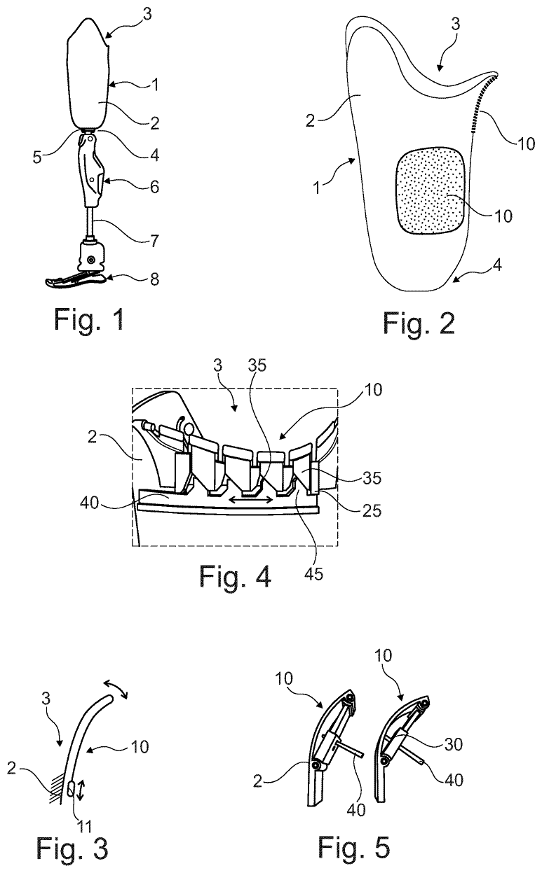

FIG. 1 shows an overall view of a prosthetic leg;

FIG. 2 shows an individual view of a prosthesis socket;

FIG. 3 shows a schematic view of a section through a socket wall;

FIG. 4 shows a plan view of a prosthesis socket with a displaceable stiffening element;

FIG. 5 shows two views of a socket edge portion in different states of stiffness;

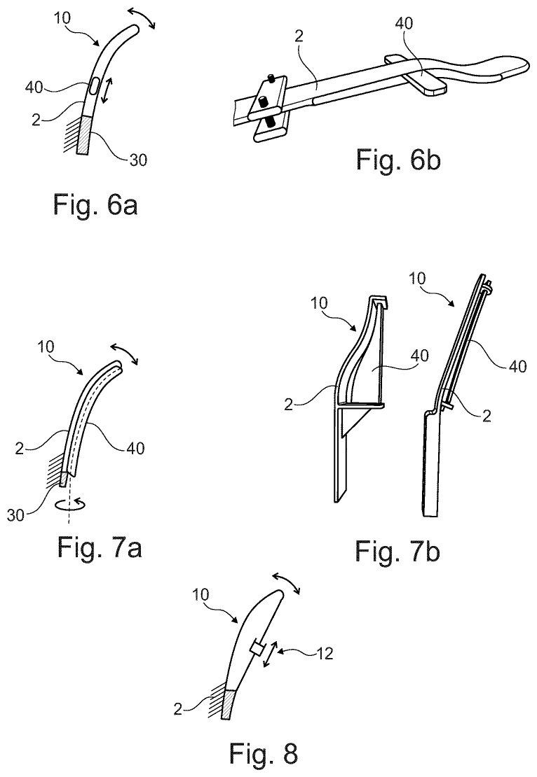

FIGS. 6a and 6b show views of a prosthesis socket portion with a displaceable stiffening element;

FIGS. 7a and 7b show views of a socket wall portion with rotatable stiffening elements;

FIG. 8 shows a sectional view of an elastic socket wall portion with a parallel damper element;

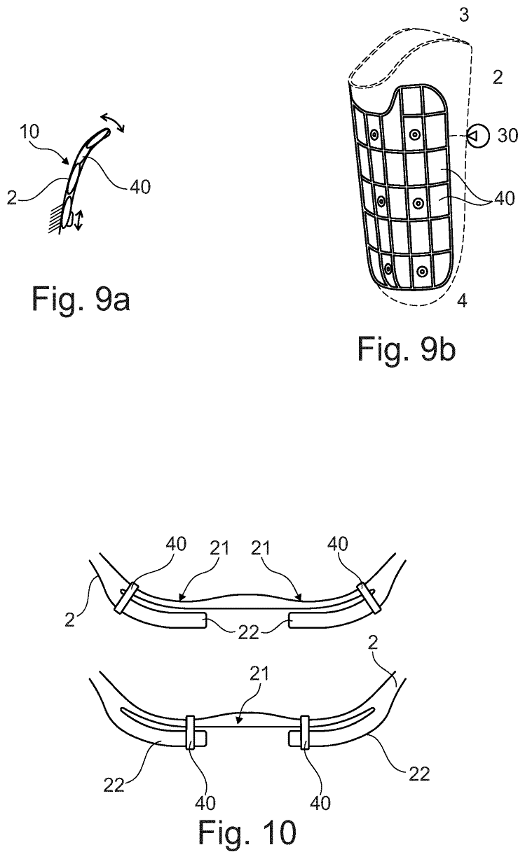

FIGS. 9a and 9b show views of a socket wall portion with volumes that can be filled and emptied;

FIG. 10 shows two sectional views with two stiffening elements;

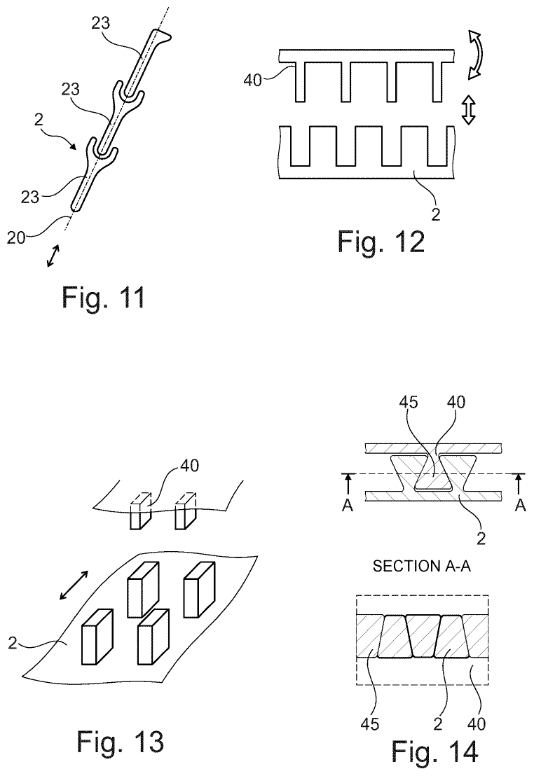

FIG. 11 shows stiffening elements displaceable in the longitudinal extent,

FIG. 12 shows a schematic view with a comb-like stiffening element;

FIG. 13 shows a view with a horizontally displaceable stiffening element;

FIG. 14 shows a variant of FIG. 13;

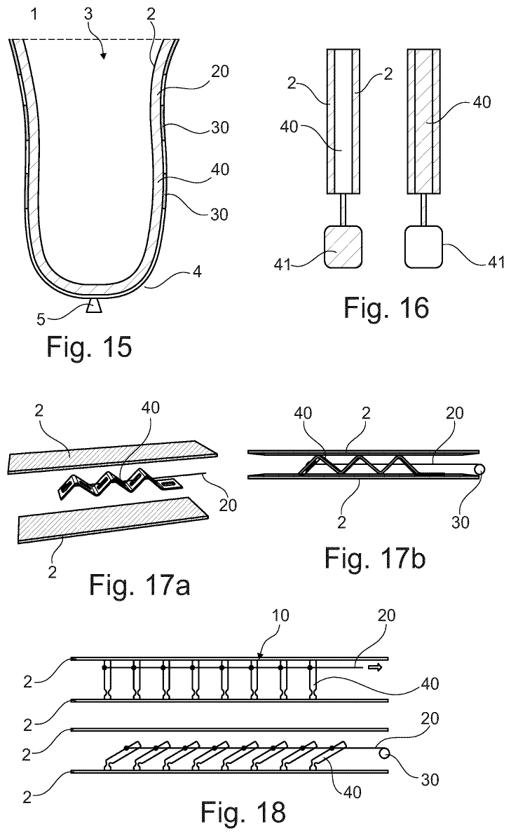

FIG. 15 shows a sectional view through a socket with magnetorheological fluid;

FIG. 16 shows a sectional view through a socket wall with a hydraulic stiffening element;

FIGS. 17a and 17b show two views with a stiffening element in the form of an accordion;

FIG. 18 shows a view of foldable stiffening elements in different positions;

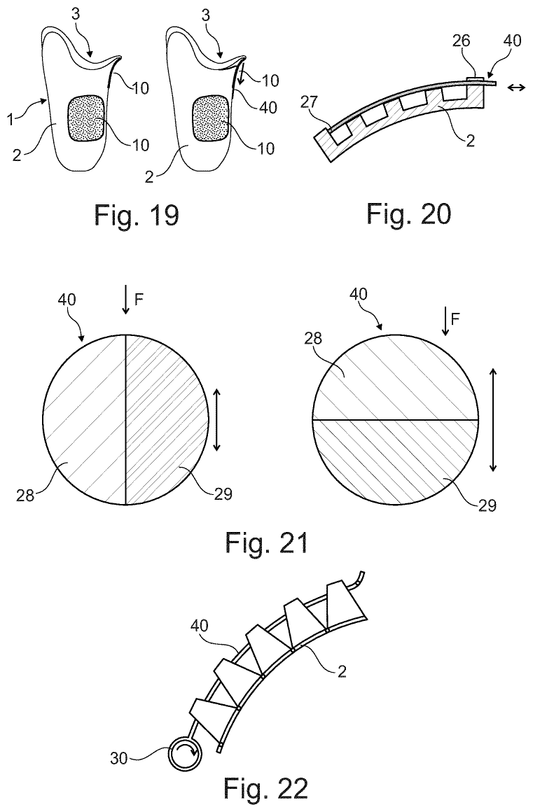

FIG. 19 shows a prosthesis socket with a displaced stiffening element;

FIG. 20 shows a socket wall portion with a stiffening element that can be tensioned;

FIG. 21 shows an embodiment of a rotatable stiffening element;

FIG. 22 shows a variant of FIG. 20;

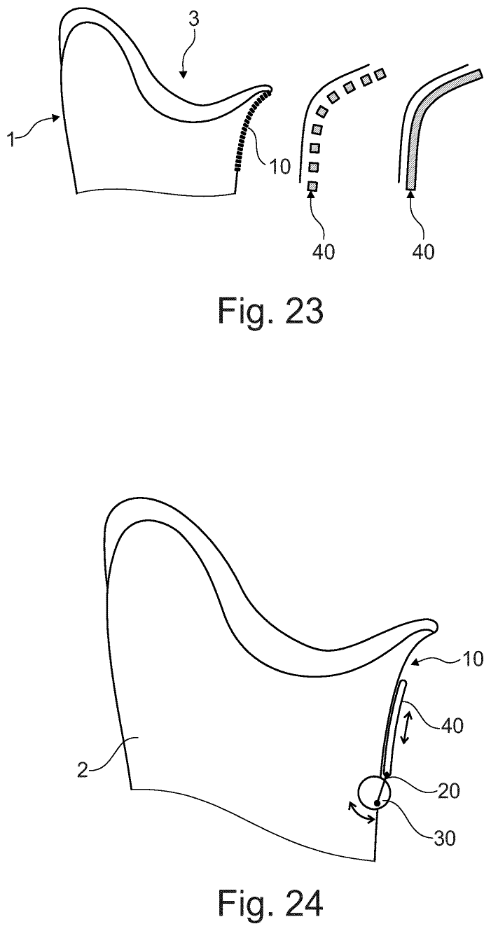

FIG. 23 shows an embodiment of a socket having a viscoelastic element with a magnetorheological fluid;

FIG. 24 shows a variant of FIG. 2 with an adjustment device;

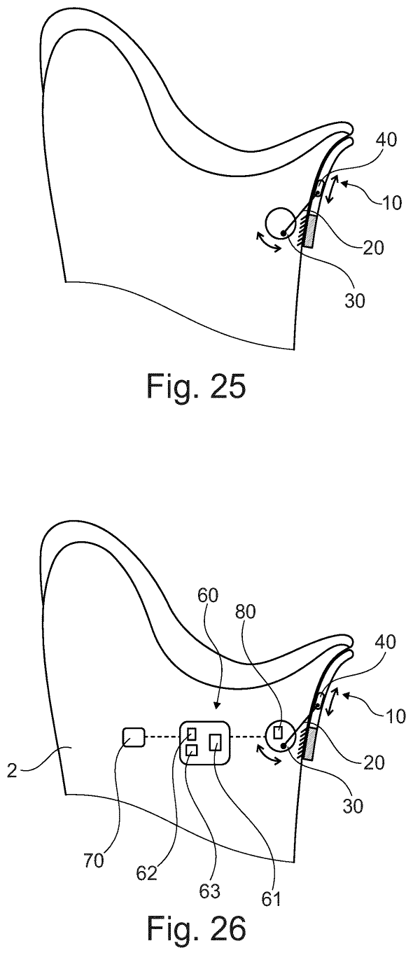

FIG. 25 shows a variant of FIG. 6;

FIG. 26 shows a variant of FIG. 25;

FIG. 27 shows an embodiment of a stiffening module;

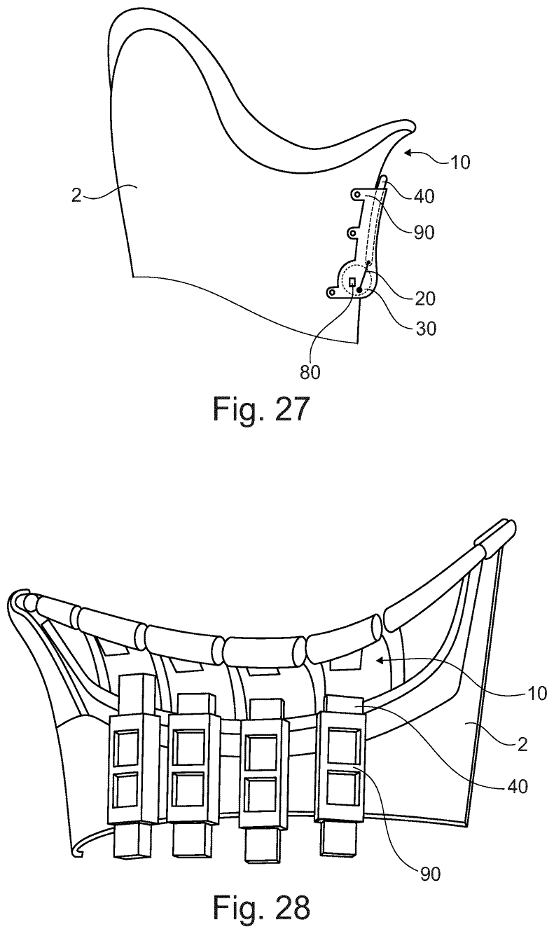

FIG. 28 shows a view of a guide for stiffening elements;

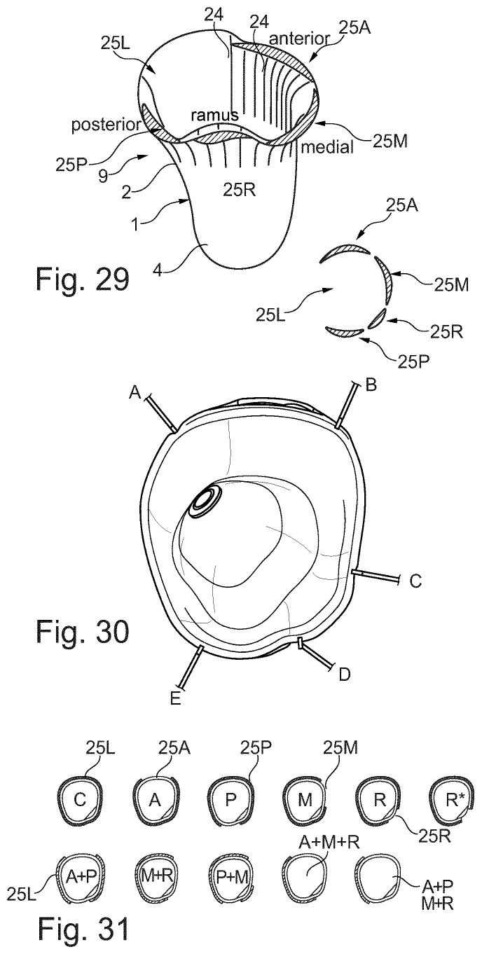

FIG. 29 shows a view of a prosthesis socket;

FIG. 30 shows a plan view of a prosthesis socket;

FIG. 31 shows possible switching;

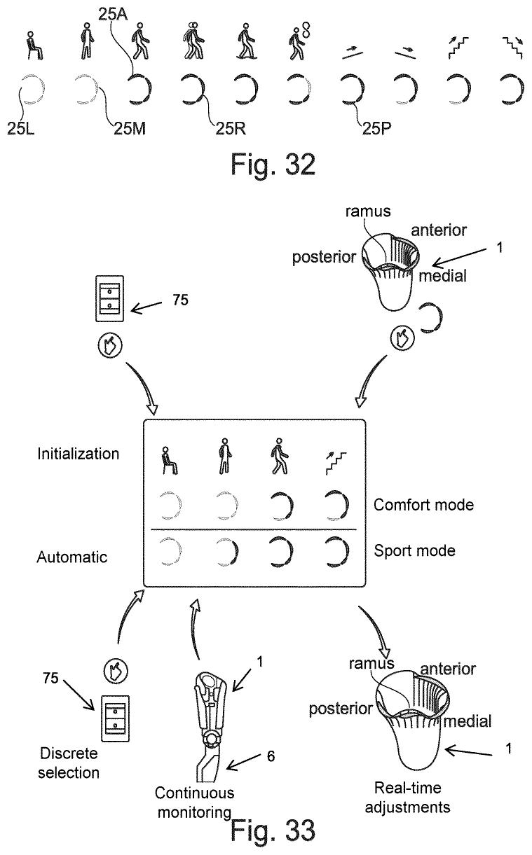

FIG. 32 shows situations with different stiffnesses;

FIG. 33 shows a control of the adjustment of the stiffness;

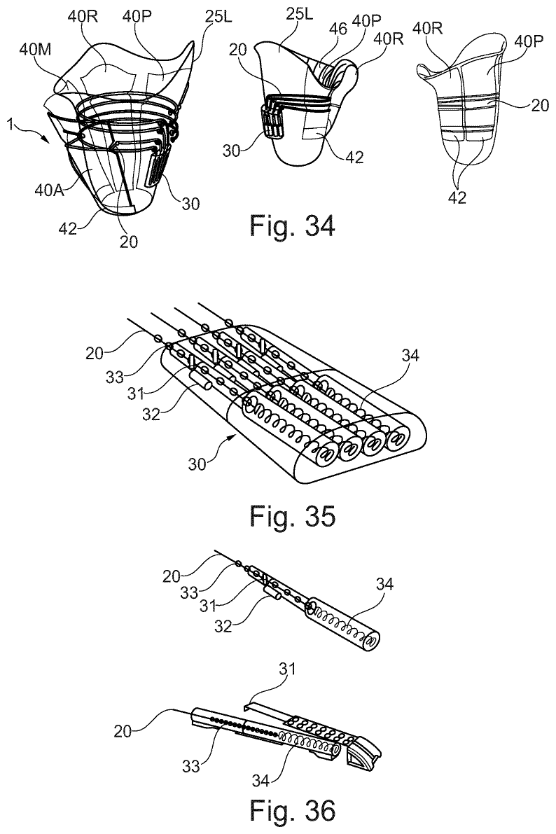

FIG. 34 shows a view of a prosthesis socket;

FIG. 35 shows an individual view of an adjustment device;

FIG. 36 shows individual views of components of an adjustment device;

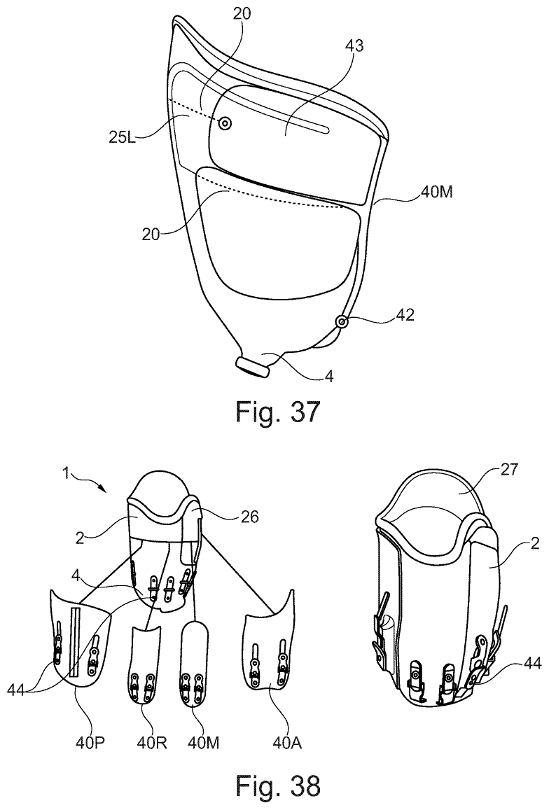

FIG. 37 shows a side view of a variant of a prosthesis socket;

FIG. 38 shows a variant of a prosthesis socket with removable stiffening elements;

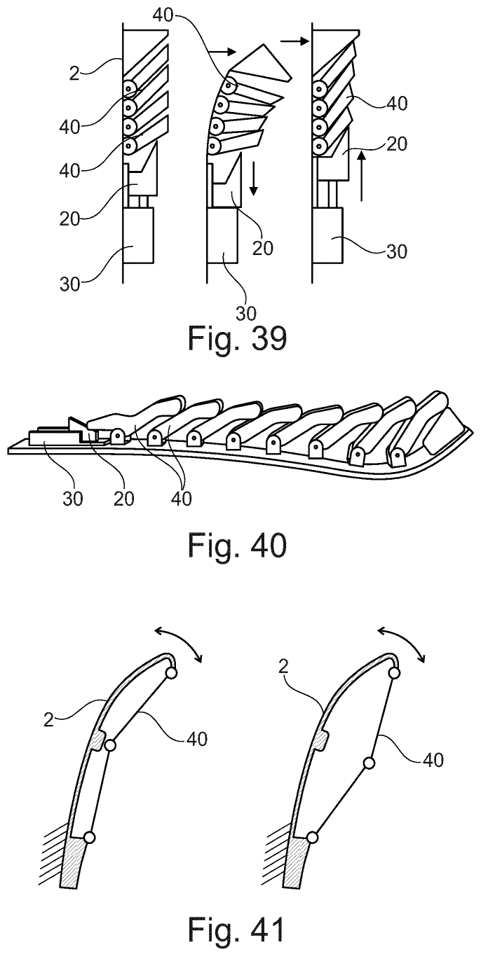

FIG. 39 shows a variant of a stiffening element with adjustment device and adjustment element;

FIG. 40 shows a variant of FIG. 39;

FIG. 41 shows a stiffening element as a toggle lever closure;

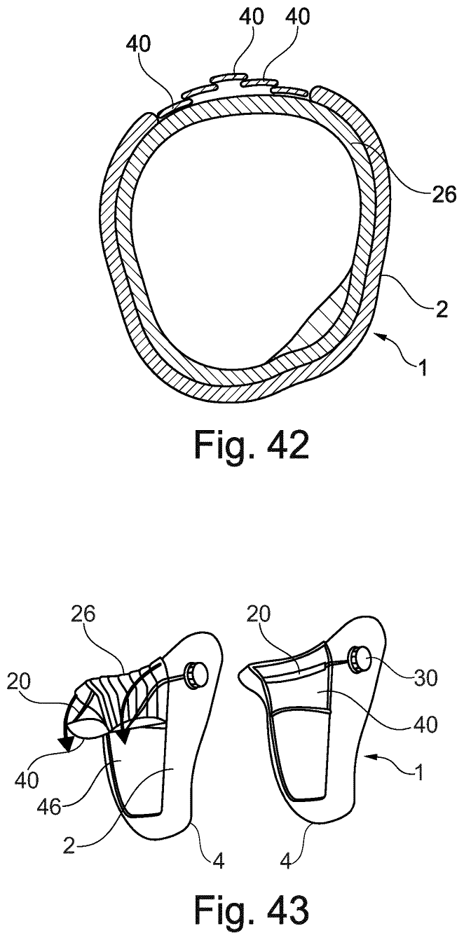

FIG. 42 shows a plan view of a variant of a prosthesis socket; and

FIG. 43 shows two views of a further variant.

FIG. 1 shows a socket 1 in the form of a prosthesis socket with a fastening device 5 in the distal end region. The fastening device 5 is designed as a so-called pyramid adapter and serves to fasten a prosthetic knee joint 6, which is coupled to a prosthetic foot 8 via a lower-leg tube 7.

FIG. 2 shows a schematic view of a socket 1 in the form of a prosthesis socket with a socket wall 2, a proximal access opening 3 and a distal end region 4. The distal end region 4 is closed in the exemplary embodiment shown and forms an end cap, at the distal end of which is arranged a fastening device (not shown) for a joint, for example a prosthetic knee joint or a prosthetic ankle joint. The socket wall consists predominantly of a dimensionally stable material and is designed with a closed cross section, so that a limb stump can be accommodated within the volume surrounded by the socket wall 2. The limb stump can be inserted into the prosthesis socket 1 and fixed therein either directly or by means of what is called liner technology, in which a flexible and elastic prosthesis liner is locked by vacuum technology or by means of mechanical locking using a pinlock.

On the lateral side, the prosthesis socket 1 is designed with an elevation of the socket wall 2 in order to enable secure guidance, while on the medial side the socket wall 2 is cut deeper in order to form the so-called ramus contact. In a central region of the socket wall 2, a first region 10 is formed in which the stiffness of the socket wall 2 can be changed, for example via a volume that can be filled and emptied, which volume is located in the socket wall 2 that is hollow there. In addition, in the region of the ramus contact, a second region 10 is formed in the socket wall, the stiffness of which region can be changed. Possibilities for adjusting the stiffness in the illustrated regions 10 and in other regions are explained in more detail below.

FIG. 3 shows a first embodiment in the form of a schematic sectional view through a socket wall 2 of a socket with the proximal edge in the region of the ramus contact. In this region, the socket wall 2 has a double spring, the foot point 11 of which is mounted in a displaceable manner. The displacement of the foot point 11 can be effected by motor or magnets. A pneumatic or hydraulic displacement of the foot point 11 is also provided in one embodiment. If the foot point 11 is shifted downward, the double spring is prestressed in the region 10 and the stiffness in the region 10 changes. The stiffening element is the double spring, which can be influenced in its stiffness by changing the position of the foot point 11 via an actuator (not shown) as an adjustment device or by manual adjustment via a lever or slide as adjustment device.

A variant of the adjustment of the stiffness in the ramus contact is shown in FIG. 4, which shows a plan view of the proximal edge region of the socket with the socket wall 2 and the proximal access opening 3. The proximal socket edge is slotted and has bevels 35 which engage with a stiffening element 40. If the stiffening element 40 is shifted to the right, for example via a cable that is wound up or via a gear drive, the bevels 45 of the stiffening element 40 are pressed against the bevels 35 on the socket wall 2 and thus pushed upward. At the same time, the wedges are pressed against the abutment 25 on account of the force component acting in the circumferential direction, as a result of which the stiffness in this region 10 increases. If the stiffening element 40 is shifted to the left, the flexibility in this region is increased.

FIG. 5 shows a variant of a socket wall 2 with a stiffness adjustable by rotation of the stiffening element 40, for example via a motor as adjustment device 30. The stiffening element 40 is moved in the direction of the socket wall 2 via the adjustment device 30, which is mounted on the socket wall 2. The adjustment device is mounted on the socket wall 2 so as to be pivotable in a proximal and distal direction and thereby has the effect that the socket wall 2 curves more strongly in the region and has a higher pretensioning and thus a higher degree of stiffness. The stiffening element 40 can also be designed to be flexible and to bring about a change in the stiffness of the socket wall 2 by changing the contact pressure via the adjustment device 30 at points or areas of the surface in the region 10. The displacement of the stiffening element 40 by the adjustment device 30 can be effected, for example, by a threaded drive, a spindle, a motor or a lever, and the fixing in the desired position is effected, for example, on the basis of a self-locking of a thread or by the clamping of the stiffening element 40 in the desired position. The stiffening element 40 acts on the socket wall 2.

FIGS. 6a and 6b show a socket wall 2 with a displaceably mounted stiffening element 40. The socket wall 2 is designed, in the region 10 with the adjustable stiffness, in the form of a double spring and has a displaceably mounted stiffening element 40 as an intermediate element, which serves to block the individual layers of the double spring. Depending on the positioning and orientation of the stiffening element 40, a changed stiffness is achieved in this region. The stiffening element 40 can be adjusted by motor via an adjustment device 30 or manually.

FIGS. 7a and 7b show an embodiment of the socket wall 2 in a proximal end region with a double spring with a rotationally adjustable single layer as stiffening element 40. Different positions of the stiffening element are shown in FIG. 6b. In the view on the right in FIG. 6b, there is reduced stiffness in comparison to the view on the left. The view on the left reveals the non-rotationally symmetrical shape of the stiffening element 40, which results in a special shape of the socket edge in the twisted state. Here too, different positions of the stiffening element 40 result in different degrees of stiffness of the socket wall 2.

FIG. 8 shows the embodiment of the socket wall 2 with the region 10, the stiffness of which is adjustable, in the form of an individual spring on which a damper element 12 is arranged. The wall region 10 executes a pivoting movement in the proximal end region, which is influenced by a longitudinal displacement of the damper element. The higher the resistance in the damper element 12, the greater the stiffness of the spring and thus the region of the socket wall 2. The damper element 12 can be designed as a hydraulic damper, pneumatic damper or solid-state damper and is adjustable, for example via a prestressing element or an adjustment device via which a valve is shifted. A solid-state damper can be compressed or relaxed via the prestressing device, as a result of which the deformation resistance can be increased or decreased. In this embodiment, the damper element 12 is the stiffening element that brings about the change in the stiffness of the socket wall 2.

FIGS. 9a and 9b show a variant with fillable volumes. The socket wall 2 is double-walled, in particular in the form of an elastic double spring, and has stiffening elements 40 within the hollow space between the two walls, which stiffening elements 40 can be filled or emptied via a pump as adjustment device 30. Depending on the degree of filling, the stiffness changes in the respective region. The pump can be designed as a hydraulic pump or as a pneumatic pump. The adjustment device 30 can be activated or deactivated manually or automatically. Automatic activation can take place, for example, on the basis of sensor data, for example spatial position data, pressure data or temperature data. The sensor data are made available to a control device, which evaluates the data and transmits an activation or deactivation command to the respective actuator.

FIG. 10 shows a detail of a sectional plan view, in which the socket wall 2 is divided into different socket wall regions. An inner, continuous socket wall region 21 extends about the circumference of a limb or limb stump (not shown). Parallel thereto, two outer socket wall regions 22 are arranged on the outside of the continuous, inner socket wall region 21, integrally molded thereon in the illustrated embodiment. These outer socket wall regions 22 extend over a certain height or a certain proximal-distal extent along the outside of the prosthesis socket on the socket wall. One or more displaceable stiffening elements 40 are likewise arranged on the socket wall 2 and, in the exemplary embodiment shown, are designed as clasps or slides which are arranged on the inside of the inner socket wall region 21 and on the outside of the outer socket wall region 22. A variable assignment of the inner and outer socket wall regions 21, 22 is carried out via the stiffening elements 40. The stiffening elements 40 can also be arranged in the space between the outer and inner socket wall regions 21, 22 and/or in grooves or guides in the socket wall regions 21, 22. The two outer socket wall regions 22 form between them a gap, so that between them only the inner socket wall region 21 is available as total material thickness of the socket wall 2 in the radial extent. In the top view in FIG. 10, the stiffening elements 40 are moved away from each other to the maximum extent, such that a mechanical association and force transmission between the outer socket wall region 22 and the inner socket wall region 21 takes place in the region of the splitting of the two socket wall regions 21, 22. As a result, the proportion of the inner socket wall region 21 that alone contributes to providing stability or stiffness of the socket wall 2 is increased. In the bottom view, the two stiffening elements 40 are shifted toward each other, such that larger regions of the outer socket wall regions 22 contribute to the absorption of forces when the socket wall 2 is loaded, thereby increasing stability and increasing the stiffness in this region. A plurality of stiffening elements 40 spaced apart in the proximal-distal direction can be provided on the socket wall 2 in order to also provide different degrees of stiffness in the proximal-distal direction via different positioning of the individual stiffening elements 40. The stiffening elements 40 can be adjusted manually by displacement; alternatively, drives of an adjustment device can be assigned to the stiffening elements 40, for example magnets, motors, pumps or the like, in order to achieve displacement along a displacement path. As an alternative to a displacement of clasp-like or slide-like stiffening elements 40, these can also be designed as clamps which pull or press the outer socket wall region 22 toward the inner socket wall region 21 in order thereby to thicken and increase the effective material thickness in this region. As an alternative to a circumferential displacement of the stiffening elements 40, these can also be designed to be displaceable in the proximal-distal direction. The socket wall regions 21, 22 are designed to be elastic, for example, such that the stiffness in this region can be changed by moving the stiffening elements 40 up and down along the longitudinal extent of the socket 1.

FIG. 11 shows a variant in which socket wall segments 23 can be displaced relative to one another in their longitudinal extent. The respective socket wall segments 23 are mounted on one another such that they can be folded or pivoted relative to one another and have corresponding bearing points which are designed in the manner of clasps in the exemplary embodiment shown. The correspondingly shaped regions of the socket wall segment 23 are drawn into or pushed out of this clasp, resulting in a wedging of the socket wall segments 23. The wedging can take place, for example, via a tension device or a push-pull rod as adjustment element 20, which can be displaced along the longitudinal extent of the socket wall segments 23. If the socket wall segments 23 are shifted toward one another and wedged together, the stiffness increases; if a reverse movement is carried out or permitted, for example by a tension means against a spring force being relaxed and the spring force or an elastic element pressing the socket wall segments 23 apart, an increased or facilitated pivotability of the socket wall segments 23 with respect to one another is permitted and the stiffness of the socket wall 2 is in this way changed in some regions.

FIG. 12 shows a further variant, in which comb-like projections or incisions are arranged on the outside of the socket wall 2. The stiffness and stability of the socket wall 2 can be changed in the free spaces between the tines or projections via a stiffening element 40 which can be moved in the direction of the socket wall 2 or removed therefrom. Corresponding free spaces and tines are arranged on the stiffening element 40, which can be inserted into and removed from the free spaces between the prongs on the socket wall 2. Depending on how far the stiffening elements 40 or tines are inserted into the free spaces, the stiffness in this region is increased or decreased.

A variant of FIG. 12 is shown in FIG. 13, in which the stiffening element 40 with the tines or projections is not displaced radially outward away from the socket wall 2, but instead a horizontal displacement takes place along the outer surface of the socket wall 2. The projections or block-like shoulders on the socket wall 2 are brought into contact with or out of contact with the projections or shoulders of the stiffening element 40 by the displacement of, for example, an adjustment element by an adjustment device. Depending on the overlap and degree of filling of the intermediate spaces between the blocks on the socket wall 2 by the stiffening element 40, the stiffness and dimensional stability of the socket wall 2 increase or decrease in this region.

FIG. 14 shows a further variant, in which knobs with a dovetail-shaped cross section are arranged or formed on the socket wall 2. Correspondingly, projections in a dovetail shape are arranged on the stiffening element 40. Thus, both projections and knob-like elements on the stiffening element 40 and on the socket wall 2 have undercuts, as a result of which an improved force transmission can take place in the illustrated state in which the components are shown. By virtue of the multiple wedge shape of the knobs or the form-fit elements, it is possible to activate the components more easily and to facilitate their insertion and engagement with one another. By virtue of the wedge-shaped configuration, a movement-dependent jump in stiffness can also be achieved. The upper view shows a section in the radial direction, while the lower view shows the section along the line A-A in the top view, which corresponds to a section in the circumferential direction.

FIG. 15 shows a further variant, in which the socket 1 is designed as a prosthesis socket. The socket wall 2 is hollow and has a magnetorheological liquid 40 as stiffening element in its interior. Electromagnets are arranged on the outside of the prosthesis socket 1 as adjustment devices 30 which act on the fluid volume. If the electromagnet 30 is activated, the viscosity of the magnetorheological liquid 40 changes, as a result of which the stiffness in the respective region is changed, in particular increased. The volumes within the socket wall 2 can be separated from one another, such that different magnetorheological liquids can be used in volumes that are separate from one another, and there are therefore structurally different changes in viscosity and thus also changes in stiffness with the same magnetic fields. The magnets 30 can also be controlled individually in order to provide magnetic fields with different field strengths, as a result of which different degree of stiffness are provided in the prosthesis socket 1 depending on the situation and location.

FIG. 16 shows a variant in which the stiffening element is designed as a cavity or cushion which is arranged as an intermediate layer between two socket walls 2 or socket wall portions. The cushion 40 is assigned a reservoir 41 in which a fluid, for example a liquid, is arranged. The fluid is moved out of the reservoir 41 into the cushion 40 and held there by means of a pump or by reducing the volume of the reservoir, for example by squeezing it by hand or by muscle activation. The socket walls 2 can be elastic; when the cushion 40 is emptied, the stiffness is comparatively low, and when the cushion is filled, the stiffness is increased. The stiffening element 40, optionally with a pump or also embedded between two leaf springs instead of the socket wall regions, can be prefabricated as a complete module and arranged and fastened on or in a socket wall. The stiffening module can thus be fastened either as an intermediate layer between two flexible socket wall regions or on an outside of the socket wall 2.

A further embodiment is shown in FIGS. 17a and 17b. FIG. 17a shows an exploded view of either a stiffening module or a socket wall with two socket wall regions 2, between which a stiffening element 40 in a corrugated shape or in an accordion-like shape is arranged. The stiffening element 40 is made, for example, from a resilient sheet metal. The stiffening element 40 is assigned an adjustment element 20 in the form of a tension means, for example a wire or a cord. FIG. 17b shows the adjustment device 30 schematically in the form of a motor which drives a roller on which the adjustment element 20 is wound. The right-hand end of the stiffening element is mounted in a stationary manner on a socket wall region, while the left-hand end can be shifted to the right via the adjustment element 20. If the adjustment element 20 is wound up via the adjustment device 30, the left-hand end of the stiffening element is displaced in the direction of the adjustment device 30, as a result of which the substantially straight portions between the bent or folded regions of the stiffening element are oriented more steeply, resulting in increased stiffness in this region. If the adjustment element 20 is relaxed, it returns to the starting position due to the elastic restoring forces of the stiffening element 40 and reduces the stiffness in this region of the socket wall 2.

FIG. 18 shows an alternative embodiment in which, instead of an accordion-like embodiment of the stiffening element 40, a plurality of foldable stiffening elements are arranged between the wall portions. In the top view, the region 10 of the socket wall is stiffened or provided with increased stiffness, while in the bottom view, with the wound-up adjustment element 20 and the adjustment device 30 as a motor drive, the stiffening elements 40 are inclined, making it easier to shift the socket wall regions 2 toward one another. The stiffening elements 40 are advantageously mounted elastically, such that, after an adjustment force by the adjustment element has ceased, they return to their original position. The adjustment element 20 can also be designed as an element that transmits tensile and compressive forces in order to bring about a pivoting movement in both directions.

FIG. 19 shows a variant of a prosthesis socket with two regions 10 whose stiffness can be changed. In addition to a planar functional region 10 in the middle of the prosthesis socket 1 within the socket wall 2, a stiffening element 40, which can be shifted via an adjustment device (not shown) or manually, is arranged at the so-called ramus contact at the proximal edge in the region of the access opening 3. The stiffening element 40 is shifted from the initial position, shown in the view on the left, into the relief position, shown in the view on the right, so that an entire region 10 at the upper edge of the socket changes in terms of stiffness. The stiffness of the prosthesis socket 1 is reduced in the upper region, for example in order to make sitting more comfortable for a socket user.

FIG. 20 shows a further embodiment in which the socket wall 2 is stiffened by means of a stiffening element 40 designed as a tension element. If the stiffening element 40, which is fixed at a fastening point 27 on the socket wall 2, is stretched on the outside over knobs or elevations of grooves or channels, the overall stiffness of the socket wall 2 increases in this state. The tension, once set within the adjustment element 20, can be fixed via a clamping device 26. If the tension element or the stiffening element 40 is relaxed, the prestressing of the socket wall region in which the tension element is effectively stretched is modulated and reduced, as a result of which the overall stiffness of the region is reduced again.

FIG. 21 shows two different orientations of a stiffening element 40, which consists of two parts 28, 29 which each have different strength or stiffness. The left half 28 is made of a soft, pliable material, while the right half 29, as shown on the left, is made of a firm, less pliable material. If a force F is exerted on the stiffening element 40 from above, for example, different degrees of resilience and stiffness result depending on the position and orientation of the two halves relative to each other. In the view on the left, the two halves act like spring elements arranged parallel to each other. On account of the orientation of the sectional plane in the middle of the stiffening element parallel to the direction of force, there is a high degree of stiffness or strength and a high resistance to displacement, which is indicated by the short double arrow. With a rotation of 90° to the orientation of the force direction F, as shown in the view on the right, there is less bending resistance and thus less strength in the region in which the stiffening element 40 is arranged. The rotation in relation to the respective direction of force can be effected manually or by motor or by another drive.

FIG. 22 shows a variant of FIG. 20, in which a tension means is likewise arranged as stiffening element 40 on an adjustment device 30, for example a roller. The adjustment device 30 can be driven manually or by motor and can be displaced in one direction or the other on the basis of a switch command or of sensor data. The end of the stiffening element 40 facing away from the adjustment device 30 is mounted on a projection on the socket wall 2 and forms an abutment, such that an increased or reduced tension and pretensioning is achieved in this region by winding or unwinding of the tension element.

FIG. 23 shows schematically a variant with a stiffening element 40 in the region 10 of the socket 1. The prosthesis socket 1 is basically elastic in this region and has an inlay or a stiffening element 40 with magnetorheological properties. The view on the right shows two states of the stiffening element 40, while the view on the left shows a non-polarized state, in which the stiffening element 40 is very flexible. In the view on the right, the magnetorheological fluid is polarized and, on account of the increased viscosity, will offer greater resistance to a load. It is thus possible for forces in this region to be countered by a greater resistance to deformation. When the load is removed and the polarization is removed, the socket wall 2 of the prosthesis socket 1 returns to the starting position that was originally set by the orthopedic technician.

FIG. 24 shows a stiffening module on the socket wall 2 with a motor-driven adjustment device 30 on which an adjustment element 20 is mounted via a crank drive. Depending on the direction of rotation, it is possible to move the adjustment element 20 in the direction of the proximal edge of the socket or in the opposite, distal direction. A stiffening element 40 is arranged on the adjustment element 20 and is mounted, for example, in a guide so as to be displaceable on the socket wall 2. The region 10 of the socket is basically designed to be flexible and can be stiffened by upward movement of the stiffening element 20, such that increased flexural stiffness is provided.

FIG. 25 shows a variant of FIG. 24, in which the stiffening module is fastened to the socket wall 2 in an exchangeable manner. Alternatively, the stiffening module can also be arranged fixedly on the outside of or inside the socket wall 2 without adjustment device 30, with the displaceable adjustment element 20 and the displaceable stiffening element 40. By rotation of the adjustment device 30, the adjustment element 20 and the stiffening element 40 are displaced via the crank drive, and the stiffness of the fixed stiffening module is changed in this region.

FIG. 26 shows a further embodiment, which substantially corresponds to that of FIG. 25. In addition to the adjustment device 30 with the stiffening element 40, which is slidably mounted along the outer wall of the prosthesis socket, a control device 60, coupled to at least one sensor 70, is shown in FIG. 26. The coupling can be made wirelessly. Alternatively or in addition, a coupling of the sensor 70 or of the sensors 70 via a cable or via another conductive connection is possible and provided. The necessary components for processing the sensor signals are accommodated within the control device 60, in particular a memory component 61, a data processing device 62 and a communication interface 63. The communication interface 63 can send data to or receive data from external devices and supply them for processing to the data processing device 62, for example a processor. In addition, the other necessary components are also integrated in the control device 60 or coupled thereto, for example an energy accumulator, possibly amplifiers or other electrical, electronic or electromechanical components.

The control device 60 transmits the necessary activation signals and/or deactivation signals to the adjustment device 30, which is coupled to an actuator 80, which is designed as an electric motor in the exemplary embodiment shown. The transmission can also take place via the communication interface 63. The actuator 80 displaces, for example, a rotary disk of the adjustment device 30 in one direction or the other, so that the adjustment element 20 in the form of a push rod shifts the stiffening element 40 in one direction or the other, in order to bring about a change in the stiffness of a region 10 of the socket wall 2.

FIG. 27 shows a further variant, in which all the components for changing the stiffness of the socket wall 2 are combined in a stiffening module. The stiffening module has a housing that can be reversibly fixed to the outside of the socket wall 2 by means of screws or rivets or other fastening elements. The stiffening module can also be embedded in the socket wall 2 or integrated into the socket wall 2. The housing of the stiffening module serves at the same time as a guide for the stiffening element 40, which in the exemplary embodiment shown is designed as a longitudinally displaceable stiffening component, for example as a slide, a deformable strip or a spring. Depending on how the actuator 80 moves the adjustment device 30 with the adjustment element 20, the stiffening element 40 is pushed up or pulled down and thereby changes the stiffness in the proximal socket wall region 10.

In FIG. 28, a total of four stiffening elements 40 are arranged in guides 90 on the outside of the socket wall 20. The stiffening elements 40 can be moved up or down, either manually or via an actuator (not shown). The further the stiffening elements 40 are shifted upward, the stiffer is the proximal socket wall region 10, which can also be subsequently inserted into the socket wall 2 and fastened thereto. For this purpose, a corresponding recess is worked into the socket wall 2 or is introduced into it, and then the socket wall region 10 is in particular permanently attached to the socket wall 2, for example via form-fit elements, fastening elements or also with cohesive bonding by welding, gluing or similar. The socket wall region 10 has slits between the individual stiffening elements 40, such that the respective regions can be changed separately from one another in terms of their stiffness.

FIG. 29 shows a further embodiment of the prosthesis socket 1 which has a peripheral socket wall 2 and a distal end region 4. The distal end region 4 and the continuous socket wall 2 form a substantially rigid, sleeve-shaped prosthesis socket 1, in the proximal edge region 9 of which recesses 24 running in the distal direction from the proximal socket edge are introduced in some regions. The prosthesis socket 1 is divided into a total of five sectors 25, of which a first, lateral sector 25L has no recesses 24. The other four sectors are an anterior sector 25A, a medial sector 25M, a posterior sector 25P, and a sector 25R for ramus contact. In the exemplary embodiment shown, except for the lateral sector 25L, all the other sectors 25A, 25M, 25R and 25P have recesses 24. The recesses 24 can have different widths in sectors, have different densities and/or different lengths. The longer the recesses 24 extend in the distal direction, the more flexible is the proximal edge region 9 in this sector. The same applies to a greater width of the recesses 24 and to an increased or high number of recesses 24 per circumferential length. Since the lateral sector 25L is mainly responsible for lateral guidance and stability, and since adjustable resilience or elasticity is not necessary there on account of the usual movements with a prosthesis socket, in most cases there are no recesses in the lateral sector 25L. As an alternative or in addition to the recesses 24, different material thicknesses can be present in the individual sectors in order to be able to adjust the resilience or elasticity of the socket entry plane or in the proximal edge region 9. In the lower right of FIG. 29, a plan view of the prosthesis socket is shown with the individual sectors 25. The orientation and designations result from the direction of walking. In the exemplary embodiment illustrated, a prosthetic socket 1 for a left stump is shown.

The individual sectors 25A, 25M, 25R, 25P and optionally 25L are designed to be individually changeable in terms of their stiffness. It is particularly advantageous here for only the proximal socket wall region 9 to be flexible and switchable in terms of stiffness, in order to have sufficient rigidity and stability in the remaining region. The division and dimensioning of the respective sectors can be done individually. An example of a division of the sectors is illustrated in FIG. 30, in which a prosthesis socket is shown in a plan view. The first reference point A is the intersection for the diagonal dimension and the lateral anterior-posterior bearing and forms the anterior end of the lateral sector 25L. The diagonal dimension is the dimension from the ramus engagement to the greater trochanter. Reference point B is the intersection of the front edge of the perineum and the medial anterior-posterior abutment; the anterior sector 25A lies between reference point A and reference point B. Reference point C lies at the ramus exit at the transition to the perineum in front of the adductor support; the medial sector 25M lies between reference points B and C. The sector 25R for the ramus contact lies between reference point C and reference point D, which forms the rear edge of the ramus engagement. The posterior sector 25P is located between reference point D and reference point E which, in mirror image to reference point D, is fixed to the walking direction. The lateral sector 25L lies between reference points A and D.

FIG. 31 shows 11 different options for switching the stiffening elements (not shown) via an adjustment device or the adjustment devices. Beginning at the top left in a clockwise direction, a complete stiffening or a maximum stiffening of the individual sectors is present, resulting in a prosthesis socket completely or maximally stiffened in the proximal edge region. With a reduction in stiffness in the anterior sector 25A, the rigidity in this region is reduced. In order to continue to guide the stump in this region and to prevent a part or region of the stump from being pinched when switching from increased flexibility to increased stiffness, the sectors to be stiffened or the proximal edge region to be stiffened are provided with an inner socket 26, which is designed to be flexible. The inner socket 26 can be designed in the shape of a cup with a closed distal region, or it can be arranged on the socket wall only in the region of the proximal edge region. Increased flexibility in the rear region is generated by reducing the stiffness in the posterior sector 25P; when the flexibility in the medial sector 25M is changed, the corresponding stiffening elements are accordingly activated or deactivated by the adjustment device in this sector, and, corresponding to this, a complete release or a maximum reduction in stiffness is effected by changing or selectively switching the stiffness in the ramus contact in the sector 25R. There are two options for this: in the last illustration in the top row of FIG. 31, the stiffness in the sector of the ramus contact 25R is minimized and the inner socket or the lining is not present or is particularly flexible, such that almost completely free mobility can be achieved, whereas, in the second last illustration in the top row, the stiffness in the sector 25R is reduced, but the inner socket is still present.

If several sectors are flexible, for example the anterior sector 25A, the medial sector 25M, the sector 25R for the ramus contact and the posterior sector 25P, there is maximum flexibility of the prosthesis socket 1, at least in the proximal edge region 9, since only the lateral sector 25L in the proximal edge region 9 still has the original, maximum stiffness. It is likewise possible to reduce the stiffness in three sectors, for example the anterior sector 25A, the medial sector 25M and the sector 25R for the ramus contact. Alternatively, it is possible for only two sectors to be selectively changed or reduced in terms of stiffness, for example the medial sector 25M in connection with the posterior sector 25P, the medial sector 25M with the sector 25R for the ramus contact or, for a lateral engagement, the anterior sector 25A and the posterior sector 25P.

FIG. 32 shows examples of some situations in which the respective sectors can be adjusted accordingly with respect to stiffness. The setting can be different for each person; in addition to a purely binary switch between “stiff” and “flexible”, different stiffnesses can be provided within the sectors for different gait situations or different situations of use. When sitting and standing, the illustrated exemplary embodiment provides that, apart from the lateral sector, which is not adjustable in the exemplary embodiment, all other sectors are set with reduced stiffness; when walking or when walking fast, the four other sectors have increased stiffness or maximum stiffness, likewise when walking on uneven terrain. The medial sector 25M is switched to flexible when walking around curves; all sectors are stiffened again when walking uphill; when walking downhill and also when climbing stairs, the posterior sector 25P is switched to soft; when walking downstairs, all switchable or adjustable sectors are again provided with reduced flexibility or increased stiffness.

An example of the setting of the different modes or stiffnesses in the region of the socket entry plane in the proximal edge region 9 is shown in FIG. 33. Two different modes are provided for four situations, for example sitting, standing, walking and going up stairs: on the one hand the comfort mode, shown in the top line, and the sport mode shown below. Both modes can be changed manually via an input device 75, such that the setting that is perceived as comfortable is available for every patient in every situation. Switching between a comfort mode and the sport mode can be automatic or done by manual switching with the input device 75. The movements of the user are continuously monitored by the sensors, in order to automatically detect changes in usage behavior or different movement situations. The monitoring is effected via sensors that can be present both in the prosthesis socket 1 and in the other prosthesis components 6 or just in the other prosthesis components. The setting of the stiffness of the sectors in the proximal edge region or the stiffness of the entire proximal edge region is advantageously effected in real time, in order always to be able to provide an adapted stiffness of the prosthesis socket 1.

An exemplary embodiment for setting sectorial or regional stiffnesses in the socket entry plane or in the proximal edge region is shown in FIG. 34, in which three views of an exemplary embodiment of a prosthesis socket 1 are shown. The prosthesis socket 1 has an inner socket 26 which completely surrounds the stump (not shown) and on the outside of which or in which a cable pull system with four cable pulls is arranged in grooves or channels. The cables are the adjustment elements 20, which are actuated by a central adjustment device 30 on the outside of the socket wall 2. Each of the four cables is arranged circumferentially or at least partly circumferentially around the prosthesis socket and has a fixed end and an adjustable end on the adjustment device 30. Each cable is guided in such a way that it activates exactly one socket wall segment as a stiffening element 40. Each socket wall segment is designed as a shell and is arranged on the outside of the prosthesis socket 1, corresponding to a respective sector. In the exemplary embodiment shown, four socket wall segments 40A, 40M, 40R, 40P are positioned, i.e. anterior, medial, in the region of the ramus contact, and posterior. The adjustment device 30 is fastened to a lateral region of the prosthesis socket 1. The prosthesis socket 1 is sufficiently stable to allow a cable to be guided on or in it in such a way that there is no change of the inner circumference or only a negligibly small reduction or deformation of the interior of the prosthesis socket 1 when the cable is tensioned. If a cable pull 20 is tensioned, the respective socket wall segment 40A, 40M, 40R, 40P, along the outside of which the cable pull 20 is guided as adjustment element, is pulled in the direction of the stump and prevents or reduces an outward lateral deformation of the prosthesis socket 1 in this region. As far as and beyond the respective socket wall segment, the respective cable runs decoupled from the socket wall segment, e.g. the cable runs within the socket wall or on the outside between the prosthesis socket and the inside of the socket wall segment.

In the view on the left in FIG. 34, the socket wall segments 40 or shells are distally connected to the prosthesis socket 1 in an articulated manner, for example via a textile or a joint 42. When the respective socket wall segment 40A, 40M, 40R, 40P is unlocked, it can lift away to a limited extent from the socket wall of the prosthesis socket 1, such that it is possible for the prosthesis socket to yield flexibly, in particular elastically, in the proximal edge region 9. The middle view in FIG. 34 shows that the prosthesis socket 1 is provided on the outside, and distally with respect to the lateral sector 25L, with the adjustment device 30, from which the four cable pulls are guided around the prosthesis socket, at least to behind the socket wall segment that is to be moved. A posterior socket wall segment 40P and the socket wall segment 40R for the ramus contact are shown; the socket wall segment 40R is released. By virtue of the joint 42 at the distal region of the socket wall segment, which joint is designed as a textile or another flexure hinge, the socket wall segment 40R can be displaced radially outward from the rest of the prosthesis socket, such that the respective region of the prosthesis socket with the sector 25R and, if necessary, with an inner socket can be shifted outward more easily when loaded by the stump than is the case with an applied socket wall segment. The view on the right shows the other two segments, an anterior socket wall segment 40A and a medial socket wall segment 40M, which both bear against the outside of the prosthesis socket 1 with the segments and recesses 24.

The socket wall segments 40A, 40M, 40P and 40R form part of an outer shell of the prosthesis socket and are actuated by the cable pulls via the central adjustment device 30, which is shown enlarged in FIG. 35. In the exemplary embodiment shown, the cable pulls, as adjustment elements 20, are prestressed via springs 34 and can be unlocked or locked via an actuator 32. On account of the springs 34, the cables 20 are held under tension on the socket. If a cable 20 is unlocked, the socket wall segment that is coupled to the cable can be pivoted outward. In the outwardly pivoted position, in which the spring 34 is tensioned, the cable can be locked, such that a permanent slackening and thus a reduction of the stiffness in the sector to which the respective segment is assigned are achieved. When the lock is released, the spring 34 contracts again and moves the socket wall segment in the direction of the stump. In the maximally shortened position of the springs 34, the effective length of the cable pull is shortened to the maximum extent, such that the socket wall segment connected thereto is maximally pressed. If the cable pull is locked in this position, the maximum stiffness in the proximal end region is set in the corresponding segment.

FIG. 36 shows two exemplary embodiments for releasing or locking the respective cable pull or the spring tension. In the top view, a tension spring 34 acts on the cable 20, to which balls 33 or projections are fastened which engage with a locking element 31. The locking element 31 is designed as a pivotable lever which, via an actuator 32, for example a magnetic switch or the like, is movable from a release position, which is shown, into a locking part, which is indicated by the arrow, and back. An alternative variant is shown in the bottom view in FIG. 36, in which a locking element 31 is moved downward from the illustrated release position into a locking position, in order to block the balls 33 or projections from moving in the direction of the longitudinal extent of the cable pull 20 or along the force action of the spring 34.

FIG. 37 shows a further variant of the prosthesis socket 1, in which flexible regions 43 in the proximal edge region are used together with rigid socket wall segments 40M, pivotably fastened to the distal, closed prosthesis socket, in order to achieve different degrees of stiffness in the proximal socket wall region. In addition to the rigid lateral sector 25L, flexible sectors 43 are provided in one exemplary embodiment in the anterior sector and the posterior sector, which flexible sectors 43 can be switched between a flexible behavior and an elastic behavior via cable pulls or other adjustment elements 20. Flexible regions 43 in the socket entry plane or in the proximal edge region offer inwardly directed flexibility. The adjustment elements 20 in the form of cable pulls are arranged within the flexible region 43 and can be tensioned or relaxed. In the tensioned state, the corresponding sector with the flexible region 43 is flexible inward and cannot be displaced outward. In a relaxed state, there is also a radial outward flexibility, in order to adapt the wearing behavior to the respective needs of the user. Such flexible regions 43 can be combined with the rigid socket segments that have been explained with reference to FIG. 34.

FIG. 38 shows an exemplary embodiment of a prosthesis socket 1 with a rigid distal end region and with four socket wall segments 40 which are rigid and which can be mounted on and detached from the outside of the prosthesis socket 1 via quick-release fasteners 44 on the socket wall 2. An inner socket 26 is arranged on the inside of the prosthesis socket 1 in order to provide a smooth-walled inner surface for receiving a stump. The lateral sector is rigid and integrally formed on the distal end region 4; the other socket wall segments 40A, 40, 40R, 40P can be attached separately to the prosthesis socket and change the stiffness in the respective region.

FIG. 39 shows a schematic detail of a further embodiment of the prosthesis socket with a socket wall 2 on which an adjustment device 30 is coupled as actuator to an adjustment element 20. The adjustment device 30 moves the adjustment element 20 upward or downward in the direction of the arrows and acts on pivotably mounted stiffening elements 40 arranged behind one another in the proximal-distal direction. In the view on the left, the stiffening elements 40 are not in contact with one another and permit mobility to the right or outward until the time or state when all the stiffening elements 40 are in contact with one another. In the middle view, the adjustment element 20 is shifted downward, and therefore the distance between the stiffening elements and the possible displacement path for the socket wall 2 are increased, such that the socket wall 2 with the upper, wedge-shaped contact element can be moved further outward. In the view on the right in FIG. 39, the adjustment element 20 is in the maximum proximal position, so that all the stiffening elements 40 are in contact with one another in this position and are also in contact with the wedge-shaped abutment element. A movement to the right or radially outward is thus prevented or at least made more difficult.

FIG. 40 shows a variant of this scale-like embodiment in which the stiffening element 40 assigned to the adjustment element 20 is designed as a double-armed lever which, on the one lever side, rests on the subsequently arranged stiffening element 40 and, on the other lever side, is changed in its position by the wedge-shaped adjustment element 20. The maximum stiffening is achieved when the adjustment element 20 has been displaced to the maximum extent by the adjustment device 30 in the direction of the first adjustment element 40. All subsequent stiffening elements 40 then lie on one another and press the proximal contact element downward, resulting in maximum stiffening. If the adjustment element 20 is shifted to the left, there is accordingly more play and flexibility in the system constructed as a module.

FIG. 41 shows a further embodiment of the orthopedic socket in a detail view, in which a stiffening element is arranged on the socket wall 2 and is designed as a toggle lever. In the view on the left in FIG. 41, the two-part stiffening element 40 bears on an abutment on the outside of the socket wall 2 and, in the event of a radially outwardly acting load, in the exemplary embodiment shown a load acting toward the right, prevents further displacement of the proximal socket edge or at least significantly hinders this. In the view on the right in FIG. 41, the stiffening element 40 is tilted outward beyond the pressure point, the central contact point on the contact element having been moved away from it, for example by a motor via an adjustment device (not shown). If a radially outwardly directed force then acts on the proximal socket wall, the two elements of the toggle lever move around the bearing arranged in the middle and enable a deformation of the prosthesis socket in this region or facilitate this deformation by virtue of the reduced stiffness.

FIG. 42 shows a sectional plan view of the prosthesis socket 1 with the prosthesis socket wall 2. An inner socket 26 is arranged on the inside and completely radially surrounds the stump. The basic structure of the prosthesis socket 1 corresponds to that of FIG. 34; other embodiments are also possible in principle. Lamellae which overlap one another are arranged in one socket wall sector, in the illustrated exemplary embodiment in the anterior socket wall sector. By virtue of the overlapping of the lamellae that form the stiffening elements 40, it is possible to manipulate the stiffness over the entire region, or within the entire socket wall sector, by manipulating a single lamella. If, for example, the central lamella is made stiff, for example with a stiffening element as shown in FIGS. 17 and 18, this stiffening element also blocks the adjacent lamellae from bending outward. As an alternative or in addition to stiffening of the individual lamellae, it is possible that the contact point of the lamellae can be sensor-controlled and reversibly changed. For example, the static friction in the overlapping regions and contact regions of the individual lamellae is changed reversibly. If a movement of the individual lamellae relative to one another is prevented, for example by differently adhering intermediate elements, by temporary covering of adhesive intermediate elements or by electromagnetic blocking of the lamellae in the overlapping region of the lamellae, the entire lamellar composite made up of the mutually overlapping lamellae is stiffened and thus serves as a stiffening element.

FIG. 43 shows a further embodiment of the prosthesis socket 1 with a socket wall 2, which likewise has a closed distal end region 4 on which fastening devices (not shown in detail) for orthopedic components, in particular prosthetic components, are arranged or formed. Within the socket wall 2 there is a window or cutout 46, in the illustrated exemplary embodiment in the anterior region, which is filled with a material or on which a cover is applied in order to provide a circumferentially closed socket wall 2 as far as the proximal end region. At the proximal end of the cutout 46, a flap is pivotably arranged as a stiffening element 40. The flap is open in the view on the left in FIG. 43, so that the inner socket 26, which has a lamellar design in this region, can be easily displaced outward. The inner socket 26 does not have to be of a lamellar design. In the view on the right, the stiffening element 40 is shifted via the adjustment device 30 and the adjustment element 20 into the closed position, moved in the direction of the stump (not shown). In this position, the prosthesis socket 1 is also substantially rigid in the proximal edge region, since the adjustment element 20, as a cable pull or belt, is flexible and substantially rigid under tension. The rest of the socket wall 2 is rigid. In a variant of FIG. 43, the socket wall 2 is designed without an inner socket 26 behind it, so that the outwardly displaced flap as stiffening element 40 increases the circumference of the prosthesis socket 1 in the proximal edge region and thereby minimizes the stiffness in this sector or region. The socket wall 2 in this region above the cutout 46 is displaceable and reduces the stiffness there to a minimum. The cutout 46 then extends as far as the upper, proximal edge and, in the proximal edge region, forms a window that can be closed or opened by the flap as stiffening element 40.

### LIST OF REFERENCE SIGNS

1 socket
2 socket wall
3 access opening
4 distal end region
5 fastening device
6 prosthetic knee joint
7 lower-leg tube
8 prosthetic foot
9 proximal edge region
10 first region
11 foot point
12 damper element
20 adjustment element
21 inner socket wall region
22 outer socket wall region
23 socket wall segment
24 recess
25 sector
26 inner socket
27 socket wall segment
28 first part
29 second part
30 adjustment device
31 locking element
32 actuator
33 ball
34 spring
35 bevel
40 stiffening element
41 reservoir
42 joint
43 flexible region
44 quick-release fasteners
45 bevel
46 cutout
60 control device
61 memory component
62 data processing device
63 communication interface
70 sensor
80 actuator
90 guide

1. An orthopedic socket for bearing on and for receiving a limb stump or a limb, comprising:
at least one socket wall;
a proximal access opening to the orthopedic socket;
a distal end;
at least one fastening device arranged for fastening at least one distal orthopedic component to the orthopedic socket; and
at least one stiffening element is arranged or formed on or in the at least one socket wall, wherein the at least one stiffening element (2), is assigned an adjustment device for adjusting a stiffness of the at least one socket wall in a is reversibly variable manner in at least in certain regions.

2. The orthopedic socket as claimed in claim 1, wherein the at least one stiffening element is mounted displaceably in or on the at least one socket wall.

3. The orthopedic socket as claimed in claim 1 wherein the at least one adjustment device is arranged on or in the at least one socket wall.

4. The orthopedic socket as claimed in claim 1 wherein the at least one stiffening element is coupled to the assigned adjustment device via an adjustment element, wherein the adjustment element is selected from the group consisting of a gear wheel, roller, crank, lever, tension means, rotary element, piston, thrust means, and valve.

5. The orthopedic socket as claimed in claim 1 wherein the at least one adjustment device is designed as an electromagnet, pump, electric motor, heater, cooling element, piezoelectric element, electroactive polymer, force accumulator or manually operable device.

6. The orthopedic socket as claimed in claim 1 wherein the at least one stiffening element is designed as a fluid volume, shape memory element, corrugated body, accordion element, toothed body, spring, foam body, phase change material, folding element, damper or electro-adhesion element.

7. The orthopedic socket as claimed in claim 1 wherein the assigned adjustment device comprises has an actuator which is activatable and/or deactivatable by a control device assigned to the actuator, wherein the assigned control device is coupled to at least one sensor or switch, wherein the at least one sensor or switch activates or deactivates the actuator based on sensor data or a switch operation.

8. The orthopedic socket as claimed in claim 7, wherein the at least one sensor or switch is designed as a temperature sensor, acceleration sensor, IMU, spatial position sensor, inclination sensor, force sensor, optical sensor, conductor electrode, pressure sensor, DMS or resistance sensor.

9. The orthopedic socket as claimed in claim 7 wherein the control device is assigned a memory, a data processing device and/or a communication interface.

10. The orthopedic socket as claimed in claim 1 further comprising a receptacle for the assigned adjustment device and/or the at least one stiffening element, wherein the receptacle is arranged in or on the at least one socket wall.

11. The orthopedic socket as claimed in claim 4 wherein the at least one stiffening element and the assigned adjustment device or the adjustment element, wherein the assigned adjustment device and the at least one stiffening element are combined as a stiffening module.

12. The orthopedic socket as claimed in claim 1 further comprising sectors with different radial elasticity formed in a proximal edge region of the at least one socket wall.

13. The orthopedic socket as claimed in claim 12, wherein the sectors comprise one or more of different materials, different material thicknesses, different widths of recesses, and a different density of recesses.

14. The orthopedic socket as claimed in claim 12, wherein the individual sectors of the sectors are assigned displaceable socket wall segments as stiffening elements.

15. The orthopedic socket as claimed in claim 1 further comprising at least one flexible region is formed in the at least one socket wall, wherein the at least one flexible region is coupled to the at least one stiffening element.

16. A method for adjusting an orthopedic socket for bearing on and for receiving a limb stump or a limb, the orthopedic socket comprising at least one socket wall, a proximal access opening in the orthopedic socket, a distal end, and at least one fastening device which is arranged for fastening at least one distal orthopedic component to the socket, comprising the step of reversibly changing a stiffness of the at least one socket wall at least in certain regions using at least one adjustment device or a stiffening element.

17. The method as claimed in claim 16, wherein the stiffness of the at least one socket wall is changed by at least one of moving an adjustment element of the at least one adjustment device, changing the stiffening element wherein the stiffening element is arranged on or in the at least one socket wall, and activating or deactivating the at least on adjustment device.

18. The method as claimed in claim 16 wherein changing the stiffness of the at least one socket wall is performed based on sensor data which are determined by at least one sensor and transmitted to a control device (60), wherein the control device activates or deactivates an actuator of the at least one adjustment device.

19. The method as claimed in claim 16 wherein changing the stiffness of the at least one socket wall is performed by twisting a spring element, moving a spring element, blocking a spring element, displacing at least one stiffening element, filling or emptying a fluid volume, changing viscosity of a medium, heating, cooling, positively engaging at least one stiffening element, wedging at least one stiffening element, and/or twisting at least one stiffening element.

20. The method as claimed in claim 17, wherein changing the stiffness of the at least one socket wall is performed by twisting, shifting, tensioning, blocking and/or activating the adjustment element.

21. The method as claimed in claim 16 wherein the at least one socket wall comprises sectors which are changed independently of one another in terms of their stiffness.

22. The method as claimed in claim 16 wherein the change in stiffness of the at least one socket wall during the use of the orthopedic socket takes place automatically, and/or as a function of a movement situation.
